(19) United States
(12) Patent Application Publication
(10) Pub. No.: US 2004/0170629 A1
(43) Pub. Date: Sep. 2, 2004

(54) Marker proteins for diagnosing smooth muscle cell abnormalities

(76) Inventor: Majed M. Hamawy, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 10/787,421

(22) Filed: Feb. 26, 2004

Related U.S. Application Data
(60) Provisional application No. 60/450,515, filed on Feb. 27, 2003.

(51) Int. Cl. G01N 33/53; G01N 33/567; G01N 33/574; A61K 39/395

(52) U.S. Cl. 424/145.1; 435/7.2

(57) ABSTRACT

Methods are provided for monitoring the condition of smooth muscles for abnormalities such as undesirable proliferation, and thereby monitoring for the presence of certain diseases such as transplant rejection, asthma and arteriosclerosis. A patient can have a sample taken from a site suspected to be subject to the disease. The sample is analyzed for the extent of presence of selenium binding protein, or phosphorylated forms thereof. Modified expression of the marker protein in the sample, or its absence, indicate the presence of such a disease. One may homogenize the sample and look for the presence or absence of a distinctly sized protein fragment resulting from the marker protein as a means of analyzing for the presence of the marker protein in the sample.

### CROSS-REFERENCE TO RELATED APPLICATIONS

This application claims priority based on U.S. provisional application 60/450,515, filed Feb. 27, 2003.

### STATEMENT REGARDING FEDERALLY SPONSORED RESEARCH/DEVELOPMENT

This invention was made with United States government support awarded by the following agency: NIH A143900. The United States has certain rights in this invention.

### BACKGROUND OF THE INVENTION

The present invention relates to methods for monitoring the condition of patients for smooth muscle cell abnormalities, particularly those associated with diseases/adverse conditions. More particularly it relates to monitoring marker proteins whose modified expression correlates with such diseases/adverse conditions.

The occurrence of transplant rejections in human patients can endanger the health of the transplant recipient. Even where the donor is a close match for the recipient, it is conventional to use drug therapy to suppress rejection episodes and/or to try to better tolerize patients to the transplanted organ. Such drugs are often quite costly. In any event, most have at least some adverse side effects. Thus, there is a desire to try to minimize the daily dosages that are administered, as well as the period of time during which the drugs are administered.

One way to try to do this is to closely monitor the condition of transplant patients as drug therapies are modified to make them less aggressive. One method is to look for indicators of the failure of the transplanted organ, such as in the case of kidneys monitoring urine output or serum creatinine levels. This approach is primarily valuable for kidney transplant patients, with much less applicability for other transplants. Further, there can be significant damage before urine output or serum creatinine level indicators will change enough to cause a reading of concern. Moreover, this technique may lead to false positives or false negatives.

There are also some blood tests and biopsy procedures that are used to try to monitor a variety of attributes that may be indicative of rejection. Again, these may lead to false positives, false negatives or have other negative attributes.

Similar problems exist with respect to accurate diagnosis and monitoring of certain other diseases/conditions such as arteriosclerosis (e.g. atherosclerosis), asthma, pregnancy complications (especially those adversely affecting the uterus), intestinal smooth muscle diseases, and certain cancers. False negatives, false positives, insufficient sensitivity, and insufficient utility at an early enough stage of the disease/adverse condition all indicate a need for improved techniques to diagnose and monitor such diseases/conditions.

In separate, unrelated, work there have been reports of the isolation and identification of certain selenium binding proteins. See generally D. Behne et al., 21 Annu. Rev. Nut. 453, 457 (2001). See also NCBI Entrez references gi/18266692 and gi/18146872 (selenium binding protein 1 and 2.

The precise function of these selenium binding proteins has not been previously identified. However, there were reports indicating that they may play a role in cancer development, as low selenium-diets have been shown to induce cancer in animal models and selenium deficiency has been implicated in cancer development in humans. However the precise role of the selenium-binding proteins 1 and 2 even in cancer development was not previously known.

In any event, there is still a need for improved means for monitoring patients for diseases/adverse conditions which involve undesirable proliferation of smooth muscle cells.

### SUMMARY OF THE INVENTION

In one aspect the invention provides a method of monitoring whether an animal is experiencing a disease and/or adverse condition involving smooth muscle cell abnormalities. The method involves analyzing a sample taken from the animal for the degree of presence of a protein selected from the group consisting of:

(a) phosphorylated proteins having at least 95 percent homology to phosphorylated SEQ. ID NO. 1 in a form in which at least a tyrosine of SEQ. ID NO. 1 has been phosphorylated;

(b) phosphorylated proteins having at least 95 percent homology to phosphorylated SEQ. ID NO. 2 in a form in which at least a tyrosine of SEQ. ID NO. 2 has been phosphorylated;

(c) proteins having at least 95 percent homology to SEQ. ID NO. 1; and

(d) proteins having at least 95 percent homology to SEQ. ID NO. 2.

In applying the above 95 percent homology tests, and the other 95% homology test of this patent, I judge homology with reference to BLAST software, using its February 2003 default settings for protein sequence homology determinations.

The animal is preferably a primate, such as a human, and the disease/adverse condition is preferably selected from the group consisting of transplant rejection, arteriosclerosis, asthma, pregnancy complications associated with the uterus, and cancer. In the case of transplant rejection, the sample could be taken from the transplanted organ/tissue/cell. In the case of monitoring for diseases/adverse conditions of the patient with respect to their own organs, the organ in question can have a sample taken from it.

In the most preferred form one examines protein fragments solubilized from a homogenate of the sample for the presence of a fragment of one of the phosphorylated proteins, which is between 20 kDa and 80 kDa in size.

In another preferred aspect the invention provides a method of monitoring whether a transplant selected from the group consisting of transplanted organs, transplanted tissues, and transplanted cells is being rejected by an animal recipient of the transplant. One analyzes a sample taken from the recipient for the degree of presence of one of the above-described proteins.

Where the transplant is a transplanted organ, the preferred organs for the methods of the present invention are transplanted hearts, transplanted livers, transplanted lungs and transplanted kidneys. The method is also likely to be suitable for use in monitoring patients who have received tissue transplants (e.g. skin grafts or blood vessel portions) or transplants of cells.

One can take a biopsy from the transplant itself as the sample to be analyzed. The method may also work less invasively, such as by using urine or serum from a kidney transplant recipient. It is also possible that differing amounts of the protein being analyzed will appear in the blood in the case of varied transplants.

In another form the invention provides a phosphorylated protein fragment in a form isolated from other proteins having a size greater than 100 kDa. The protein is between 20 and 80 kDa in size and is selected from the group consisting of a fragment of phosphorylated SEQ. ID NO. 1 in a form in which at least a tyrosine of SEQ. ID NO. 1 has been phosphorylated and a fragment of phosphorylated SEQ. ID NO. 2 in a form in which at least a tyrosine of SEQ. ID NO. 2 has been phosphorylated.

Such fragments can be used to develop antibodies to the phosphorylated protein in the sample, with those antibodies in turn being useful in the analysis of the above methods. For example, such protein fragments can be used to obtain polyclonal or monoclonal antibodies by techniques well known in the art. The resulting antibodies may be tagged with a label, and become an easier and more accurate way of either directly analyzing samples for protein level, or alternatively analyzing the homogenates for level of a fragment of the protein (apart from the current gel visualization method).

While one important utility of the invention is believed to be in connection with diagnosing humans, the invention may also have veterinary applicability. Also, the invention should work regardless of whether more than one or two tyrosines have been phosphorylated in the marker protein, and also regardless whether in addition other amino acids in the protein have been phosphorylated.

If desired, the analyzing step may involve comparing the degree of presence of the phosphorylated protein with the degree of presence of the phosphorylated protein in a known standard. For transplant monitoring the standard could be one developed from a known tolerant recipient, or developed from a known rejecting recipient. Similarly, for other disease states/adverse conditions, the standard can be developed for a patient known not to have the state or condition, or can be of a patient known to have the state or condition. However, in the current most preferred form the analyzing step preferably involves determining whether a particular size fragment of the protein can be visualized at all on a gel.

The present invention provides methods for more reliably monitoring certain diseases/adverse conditions involving smooth muscle cell abnormalities. The term “abnormalities” in this context means undesirable modified (usually increased) levels of proliferation, undesirable differentiation, and/or undesirable function, of smooth muscle cells. For example, elevated levels of proliferation are central to the rejection or tolerance status of a transplant recipient. Thus, monitoring works for monitoring transplant status. It should assist a treating physician in knowing when further reductions of drug levels are advisable or inadvisable, as well as send an early warning when an adverse medical condition is beginning to take hold.

In any event, the present invention should provide a back-up or confirmatory test, even when it is not preferred as a preliminary screening test. This may be particularly valuable when the screening test is something like urine output, variations in which may have many other possible causes.

Smooth muscle cells are found in essentially all blood cells. Moreover, they are also found in the lung, uterus and intestine.

In other aspects the invention provides antibodies and kits for practicing the above methods.

These and still other advantages of the present invention will be apparent from the description which follows. The following description is merely of the preferred embodiments. Thus, the claims should be looked to in order to understand the full scope of the invention.

### DESCRIPTION OF THE PREFERRED EMBODIMENTS

A. Overview

Allograft vasculopathy (allograft arteriosclerosis) plays an important role in allograft rejection and is characterized by the thickening of the intima that causes ischemia and late graft failure. Intimal thickening results, at least in part, from the migration of smooth muscle cells (SMC) into the intima from the layer beneath the intima, proliferation of resident or migratory SMC, and the elaboration of extracellular matrix by SMC.

I have discovered that selenium binding protein (especially in the form of phosphorylated selenium binding protein-1), while amply expressed in normal primate smooth muscle sites, is absent or strongly expression modified (downregulated) in SMC with elevated levels of smooth muscle cell proliferation (e.g. during vasculopathy). I therefore identify selenium binding protein as a marker for SMC in vivo, and its modifications in expression thereof as a marker of pathogenesis of when SMC cells are participating in a disease state.

I used rats, mice, baboons, and rhesus monkeys in most of my experiments reported hereafter. I also examined some human cells, primarily with respect to confirming locations where various selenium binding proteins exist. In any event, the non-human animal models are believed to be good predictors of human results, particularly the rhesus and baboon models.

B. Rodent Experiments

I first took homogenates of kidney from 38 normal rats and subjected these homogenates to a series of 30, 50, and 100 kd cutoff filters. The resulting soluble fractions were then subjected to SDS-PAGE 2-dimensional gel electrophoresis, transferred to a membrane, and then immunoblotted with antiphosphotyrosine antibody. These naive kidney homogenates all contained fragments from a phosphorylated marker protein, the fragments being also phosphorylated and being between 20 and 80 kDa (about 35 kDa when we used a mild Triton buffer, and about 55 kDa when we used a more stringent urea buffer) which bound to the antibody to provide a visual presence clue.

Similar analysis was then run on kidney homogenates from six rats that had received a transplanted kidney and which were known by other measures to be rejecting their kidney. The fragment of the phosphorylated marker protein was missing from the gel for all six. These tests indicate that the reduction and/or absence of the phosphorylated protein, as monitored by examining protein fragments from it, is a marker of rejection status.

I then conducted time course studies showing the presence or absence of a band corresponding to a fragment of the phosphorylated protein marker on a polyacrylamide gel from samples taken at various times after transplant (in the absence of drug therapy). Day 3 samples showed normal band presence. Day 4 samples showed a reduced band presence. Day 5 samples showed only a trace of the band. I know that rejection episodes in this type of rat for this organ typically occur between days 4 and 6.

I then ran homogenate experiments on rats that had received drug therapy to reduce rejection episodes (e.g. FK506 or cyclosporin A for a week to ten days to induce tolerance). At a month past transplant I ran samples taken from these rates and saw a strong band indicating presence of the phosphorylated protein in all but one.

I then checked the condition of the one “suspect” rat. It turned out that the suspect rat was in fact suffering from acute rejection even though it had been undergoing drug therapy to suppress rejection.

I then also looked at mice and via similar experiments found the presence of the phosphorylated protein in that specie as well (in the normal mouse). I next confirmed the presence of the protein in normal baboon and rhesus, but marked reductions in the band where the primates were undergoing rejection.

To try to identify the marker proteins, a protein band was excised from the gel and analyzed by mass spectrometry. The results (and our later experiments) indicated that the protein fragments contain a phosphorylated form of selenium-binding protein. Two known sequences given for selenium-binding protein(s) are SEQ. ID NO. 1 and SEQ. ID. NO. 2. In accordance with the present invention, I therefore use the presence or absence of a phosphorylated form of these proteins to monitor rejection and/or tolerance.

Because the level of the phosphorylated protein decreases in rejecting animals, it is possible that these proteins are being shed out of the kidney and into the urine in kidney transplant situations. If so, sampling the levels in the urine (as distinguished from a liver or kidney homogenate) might well prove a less invasive way to practice the invention. In any event, one can take a biopsy of the transplanted organ and examine that sample.

C. Baboon Experiments

I then obtained kidneys directly from naive and (alternatively) rejecting baboons, which were placed immediately into ice-cold RPMI medium (Cellgro). Each organ was chopped into small pieces. The organ pieces were then added to cell strainers, which were placed in the wells of a 6-well tissue culture plates. Ice-cold urea buffer (12 g plusone urea-Amersham; 15 ml ddH₂O; 1 g plusone CHAPS-Amersham; 500 pl Pharmalyte 3-10 for IEF-Amersham) containing 1% PMSF (Sigma) and 1% Aprotinin (Sigma) was added to the pieces at a ratio of 1 gram tissue: 2 ml of urea buffer.

The pieces were then sieved (pushed) through a cell strainer with a syringe plunger. All reagents and lab ware were cold, and tissue processing was done as fast as practical. The lysates were collected in tubes and then centrifuged at high speed (14000 rpm for 30 minutes at 20° C.). The supernatants were transferred into fresh tubes and recentrifuged.

After centrifugation, the supernatants were filtered with 100 kDa MWCO filter (Millipore). This usually took up to two days. The filtered sample (the soluble pass through material) was concentrated in a 50 kDa MWCO Millipore filter. The concentrated sample having a size larger than 50 kDa was washed at least 3 times with fresh urea buffer containing 1% PMSF and 1% Aprotinin (at least 2 ml/wash).

After washing, the filtrates were brought up to the desired volume with urea buffer and transferred into fresh Eppendorf tubes. The tubes were stored at −20° C. until analysis.

Samples were subjected to two-dimensional electrophoresis (IEF and SDS-PAGE). The gels were then either stained with Coomassie blue or silver staining, or transferred to PVDF membranes for blotting with anti-phosphotyrosine antibodies (e.g. the PY20 antibody from BD Transduction Lab). My results showed an extremely strong correlation between the presence of the phosphorylated protein in the sample (as confirmed by the protein fragment band of about 55 kDa), and whether the donor of the sample was experiencing rejection.

D. Rhesus Experiments

I then conducted a series of rhesus monkey experiments by subjecting tissue lysates to 2-D electrophoresis, transferring the proteins to membranes and then immunoblotting with anti-phosphotyrosine antibodies (Ab). Fifty kDa proteins that were constantly present in naive but not in rejected kidneys were identified by mass spectrometry as derived from phosphorylated selenium binding protein-1 (SBP-1).

Immunohistochemistry showed phosphorylated SBP-1 to be localized in blood vessels, and moreover phosphorylated SBP-1 was smooth muscle specific. However, in the uterus phosphorylated SBP-1 was detected in vascular SMC and uterine SMC, indicating that this protein is not specific to vascular SMC. Phosphorylated SBP-1 was also detected in the lysates of aortas.

Rhesus kidney lysates were filtered and concentrated using 100 kDa and 50 kDa molecular weight cut-off filters, subjected to 2-D electrophoresis and Western transfer, and then the proteins blotted with anti-SBP-1 Ab. The anti-SBP-1 Ab was stripped off and the membranes were reimmunoblotted with anti-phosphotyrosine Ab.

Identical spots were detected by both Ab, indicting that SBP-1 is tyrosine phosphorylated in vivo. To confirm these results we examined the phosphorylation of SBP-1 by mass spectrometry. In-silica digest of SBP-1 was performed using the UCSF Protein Prospector program. Protein tyrosine phosphorylation was a considered modification. The in-silica digest-generated masses were compared to the 19 masses obtained through mass spectrometry. Two masses showed tyrosine phosphorylation modification. Tyrosine 12 and tyrosine 335 were phosphorylated.

To confirm the applicability of our rodent and baboon findings, tissues from naive and from rejected rhesus monkey kidneys were subjected to 2-D gel electrophoresis and the proteins were transferred to membranes and then immunolabeled with anti-SBP-1 antibodies. SBP-1 was strongly expressed in the lysates of naive kidneys but not in kidneys with chronic rejection. In the rhesus monkeys SBP-1 was strongly detected in the SMC of the normal vessels, but barely detectable in vessels with vascular chronic and acute rejection.

To further analyze these results, biopsies from seventeen kidney rhesus allografts showing signs of vasculopathy were immunohistochemically labeled with anti-SBP-1 Ab and a semiquantitative scale from 0 to +3 was used to grade the intensity of vascular SMC staining. SBP-1 staining was significantly weaker in rejected kidneys compared to kidneys from naive animals. The signal was almost completely absent in specimens with severe acute and chronic rejection.

The inverse relationship between allograft vasculopathy and the level of phosphorylated SBP-1 in SMC implicates SBP-1 in the pathogenesis of allograft vasculopathy. The localization of SBP-1 to SMC and its absence in vascular rejection suggest that this protein will serve as a marker for SMC vasculopathy.

E. Most Preferred Methods

The most preferred primate materials and methods were as follows:

Cell lines. The normal human vascular (aorta) SMC line, CRL-1999, was obtained from ATCC (Manassas, Va.). CRL-1999 cells were maintained in F12K Kaighn's Modification media containing 10 mM TES, 0.3 mM L-ascorbic Acid, 0.001 mM insulin, 0.001% Apo-transferrin, 58 nM sodium selenite, 0.003% endothelial growth supplement, 1% antibiotic/antimycotic, 1% L-Glutamine, 1% HEPES, 1% non-essential amino acids, 1% sodium pyruvate, and 10% heat-inactivated FCS.

Antibody Production and Purification. Rabbit sera to phosphorylated SBP-1 were generated by ProSci, Inc. (Poway, Calif.) and Biosource International, Inc. (Camarillo, Calif.). Rabbits were immunized with a cocktail of 3 peptides corresponding to different regions of SBP-1 in Complete or Incomplete Freund's adjuvant. These peptides were fourteen or fifteen amino acid peptides beginning with cysteine. Peptides were conjugated to a standard carrier protein at a 5 mg peptide:3 mg carrier ratio.

Complete Freund's adjuvant contained 200 ug of peptide-carrier protein conjugate and was administered to each rabbit for the first immunization. Incomplete Freund's adjuvant contained 100 pg of peptide-carrier protein conjugate and was administered to each rabbit for the remainder of the immunization protocol. Anti-peptide response (titer) was monitored by ELISA, in which the plates were coated with the three peptides. Polyclonal antibodies to each individual peptide were affinity purified using peptide-linked beads.

Antibodies were eluted from the column with 100 mM glycine buffer pH 2.5 and immediately neutralized with 1 M Tris-HCl pH 9.5. The antibodies were dialyzed against borate buffer and stored in the same buffer. Both anti-peptide 1 and 2 were immunogenic and mg concentrations of antibodies were obtained. In contrast, peptide:3 was not immunogenic.

Tissue Processing and 2-Dimensional (2-D) Gel Electrophoresis. Tissues were collected in ice-cold RPMI and minced into small pieces using sharp scissors. The minced tissue was immediately homogenized in urea buffer (8 M urea, 100 mM CHAPS, 2% Pharmalyte 3-10 for IEF, 1% PMSF and 1% Aprotinin) at a ratio of 1 gram of tissue to 3 ml of Urea buffer. Lysates were then centrifuged at room temperature for 30 minutes.

After centrifugation, supernatants were collected and centrifuged for an additional 30 minutes at room temperature. After the second centrifugation, the supernatants were filtered in 100 kDa molecular weight cut-off filters and the flow-through samples were transferred to 50 kDa molecular weight cut-off filters. The samples were then washed with urea buffer and concentrated in the 50 kDa molecular weight cut-off filters down to 30 ml.

Rehydration Solution (8 M urea, 50 mM CHAPS, 0.3% Bromphenol Blue, 3 mM DTT, and 0.08% IPG Buffer pH 3-10) was combined with the samples at a ratio of 4:1, and used to rehydrate Immobiline Strips pH 3-10 (Amersham Biosciences, Piscataway, N.J.) overnight at room temperature. The following day, the Immobiline Strips were subjected to Isoelectric Focusing (1-D) using the Multiphore II system (Amersham Biosciences). After all programs had been completed, the Immobiline Strips were removed, and placed in SDS equilibration solution I, (5% Tris-HCL pH 8.8, 6 M urea, 35% glycerol, 10% SDS, 0.2% Bromophenol Blue, and 3 mM DTT), for 15 minutes.

After incubation, the Immobiline Strips were removed and placed in SDS equilibration solution II (5% Tris-HCL pH 8.8, 6 M Urea, 35% glycerol, 10% SDS, 0.2% Bromophenol Blue, and 7 mM Iodoacetamide) for an addition 15 minutes. Immobiline Strips were then placed on the top of 10% Tris/Glycine SDS-PAGE, and held in place with 0.5% agarose overlay. The proteins in the strips were then subjected to SDS-PAGE (2-D) and then transferred to PVDF membranes. Tyrosine phosphorylated proteins were detected by blotting with horseradish peroxidase (HRP)-conjugated anti-phosphotyrosine antibodies PY-20 (BD Bioscience, San Diego, Calif.) or PY69 (BD Bioscience, San Diego, Calif.). SBP-1 and actin were detected by blotting with specific primary antibodies followed with HRP-conjugated secondary antibodies. Proteins were visualized using the LumiGLO kit (KPL, Gaithersburg, Md.) according to the manufacturer's recommendations.

Cell Solubilization. Cells from adherent and non-adherent cell lines were washed three times with ice-cold PBS and then suspended in PBS at 5×10⁵ cells/ml. The cells were then lysed with ice-cold 1× lysis buffer (150 mM NaCl, 0.2% EDTA, 1% Tris-HCL, 12 mM Deoxycholic Acid, 1% Triton X-100, 0.5% SDS, plus 0.5% Aprotinin, 0.5% PMSF, 0.05% Pepstatin and 0.01% Leupeptin; final concentration). Samples were vortexed vigorously for 30 minutes while on ice.

Cell lysates were centrifuged at 4° C. for 30 minutes. Supernatants were removed, and filtered through 100 kDa molecular weight cut-off filters and the flow-through was then concentrated in 30 kDa molecular weight cut-off filters. An equal volume of 2× sample buffer (2% SDS, 100 mM Tris-HCl pH 6.8, 1% glycerol, 100 mM DTT; final concentration) was added to the concentrated supernatants, and boiled for 30 minutes.

Meanwhile, the pellets were solubilized in 1× sample buffer and boiled for 30 minutes. Supernatants and pellets were subjected to SDS-PAGE. Proteins were transferred to PVDF membranes and then blotted with specific antibodies.

Mass Spectrometry. Proteins from 2-D gel electrophoresis were visualized by Coomassie Blue staining. Desired protein spots were excised from the gel and sent to the University of Wisconsin-Madison Biotechnology Center (Madison, Wis.) for Mass Spectrometry. Samples were placed in siliconized Eppendorf tubes, washed 10 times in ddH₂O for 10 minutes, and then destained with 100 mM (NH₄)HCO₃/50% methanol.

The samples were dehydrated for 10 minutes with 25 mM (NH₄)HCO₃/50% acetonitrile, dried for 10 minutes in the vacuum centrifuge, and then rehydrated with fresh 100 mM dithiothreitol in 25 mM (NH₄)HCO₃ at 56° C. for 30 minutes. After the samples cooled, 55 mM iodoacetamide in 25 mM (NH₄)HCO₃ was added to the samples and placed in the dark for 30 minutes at room temperature. After incubation, the reaction was stopped by adding 25 mM (NH₄)HCO₃ over 20 minutes.

The samples were then dehydrated for 10 minutes with 25 mM (NH₄)HCO₃/50% acetonitrile, vacuum dried for 10 minutes, and then digested for 15 minutes at room temperature with 20 ng/ml Sequence Grade Modified Trypsin (Promega, Madison, Wis.) in 25 mM (NH₄)HCO₃ pH 8-8.5. After digestion with trypsin, the samples were overlaid with 25 mM (NH₄)HCO₃ and incubated for 16-24 h at 37° C. After incubation, the supernatant was transferred to siliconized Eppendorf tubes, and the peptides were extracted with 0.1% trifluoroacetic acid.

The extracted peptides were subjected to two additional extractions with 5% triflouroacetic acid/70% acetonitrile. Peptides were dried in a vacuum centrifuge, and then subjected to MALDI-TOF mass spectrometry on a Bruker Biflex III (Bruker Daltonics, Billerica, Mass.). The samples were run on an alpha-cyano, 4-hydroxy cinnamic acid (CCA) matrix under Reflector mode. All sample masses 900 through 3200 Da were considered. Contaminant and blank gel masses were then removed from the list. Masses were analyzed using Matrix Science Mascot Peptide Mass Fingerprint (www.matrixscience.com). The program setting was set to trypsin, allowing up to one missed cleavage, oxidation of methiodine for fixed modifications and carbaminomethylation of cysteine for variable modifications.

Peptide tolerance was set to 0.5 Da, MH+, and monoisotopic. Locating phosphorylated proteins was accomplished by performing in-silica digest on SBP-1 using UCSF Protein Prospector program (MS Digest), and considering tyrosine phosphorylation as a modification. The in-silica digest-generated masses were compared against the 19 masses obtained through mass spectrometry.

Graft Transplant. Male juvenile rhesus monkeys weighing from 2.5 to 3.5 kg (age range, 2-3 years) were purchased after a negative screening for herpes B virus, simian immunodeficiency virus, simian T-lymphotropic virus, simian retrovirus and tuberculosis. Donor-recipient pairs were selected on the basis of cytotoxic T-lymphocyte and mixed lymphocyte culture responses and major histocompatibility complex class I and II typing.

The donor left kidney was transplanted into recipients that had undergone bilateral native nephrectomy. The anti-CD3 Immunotoxin FN18—CRM9 was administered to the monkey. This therapeutic strategy prevents acute but not chronic graft rejection. Most of the animals therefore develop signs of chronic allograft nephropathy, including severe interstitial fibrosis, tubular atrophy, chronic transplant glomerulopathy, and chronic vascular rejection within 18 months after transplantation.

Graft function was monitored by measuring serum creatinine levels, and rejection was biopsy-confirmed. Animals included in this study underwent at least five serial renal allograft biopsies to evaluate graft histology, and all had graft survival of at least 100 days off all immunosuppression.

Light microscopy. Allograft core biopsies and necropsy tissues, and tissues from experimental animals were obtained and either fixed immediately in 10% neutral buffered formalin or snap frozen. Routine light microscopy on hematoxylin and eosin (H&E) stained sections were performed on paraffin-embedded tissues. The Banff-97 criteria of kidney transplant pathology was used for scoring the presence and degree of rejection.

Acute cellular rejection was defined by the presence of focal or diffuse interstitial inflammation (and edema) associated with mononuclear inflammatory cell invasion into the tubular epithelium (tubulitis) and/or intima of vessels (vasculitis). Chronic rejection (CR) was defined by the presence of interstitial fibrosis, tubular atrophy, allograft glomerulopathy, mesangial matrix increase and vascular fibrous intimal thickening.

Immunohistochemistry. Immunohistochemical labeling was performed on snap frozen specimens and on selected paraffin-embedded tissues. For frozen sections, five-micron sections were obtained from each tissue block and fixed in cold acetone and stored at −20° C. The slides were then dried at 37° C. for 30 minutes and fixed in acetone for 5 minutes at room temperature. Selected tissues were processed for routine paraffin embedding.

Five-micron sections were obtained from each tissue block, deparaffinized in xylene, and rehydrated through graded ethanol to water. The slides were subjected to heat-induced epitope retrieval in 10 mM EDTA solution using a decloaking chamber (Biocare Medical, Walnut Creek, Calif.) at 6 PSI for 45 minutes. The slides were rinsed with TBS/Tween and the nonspecific sites were blocked by using a casein-based blocking agent (Sniper, Biocare Medical). Purified polyclonal rabbit antibodies against two different peptides of SBP-1 were used to incubate the slides for one hour at room temperature.

After rinsing with TBS/Tween, the slides were treated with synthetic polymer Envision Plus HRP system (DakoCytomation, Carpinteria, Calif.). After incubation, the slides were washed with TBS/Tween and incubated with DAB chromagen (DakoCytomation, Carpinteria, Calif.) for five minutes. Following incubation, they were counterstained with hematoxylin, dehydrated, cleared and cover-slipped.

Reverse transcription polymerase chain reaction (RT-PCR). 1×10⁵ CRL-1999 cells in F12K Kaighn's Modification media were plated in 100 mm tissue culture dishes for 24 h at 37° C. After incubation, 5 ng/ml TGF-β, 1 ng/ml TNF-α, 50 ng/ml IFN-γ, or 10 ng/ml PDGF were added to the dishes and the incubation resumed for indicated time at 37° C. The cells were then detached from the plates with trypsin, washed in PBS, counted, and total RNA was extracted using the SV Total RNA Isolation System (Promega, Madison, Wis.). The resultant mRNA was reverse transcribed to generate first-strand cDNA using avian myeloblastosis virus (AMV) reverse transcriptase. SBP-1 cDNA was then amplified using PCR with the SBP-1-specific oligonucleotides, sense and antisense. The anti-sense primer corresponds to the 3′-untranslated region of SBP-1. PCR products were then subjected to 1% agarose gel electrophoresis and visualized by ethidium bromide staining.

Statistical analysis. The biopsies were divided into two categories: normal and rejection. To examine differences in staining scores across diagnostic groups, a nonparametric one-way ANOVA was performed by analyzing the ranked Pep2 staining scores. Statistical analysis was performed using SAS v. 6.12 for Windows.

When performing the methods on a human, a biopsy or other sampling will be conducted in a known manner. Thereafter, the methods will proceed in an analogous manner to that used for the above preferred protocols.

E. TGF-β Experiments

I reasoned that the decrease in the level of SBP-1 type proteins in the SMC of rejecting grafts might be due to a growth factor(s)-induced signal. I then examined the effect of TGF-β, IFN-γ, TNF-α, and PDGF on the expression of SBP-1 for a primary SMC line. Under the conditions used in our studies, only TGF-β caused a sharp decline in SBP-1 mRNA. Hence, I believe that changes in the level of selenium binding protein type proteins are due to a growth factor induced signal.

F. Other Experiments/Theory Of The Invention

The invention therefore provides a means of closely monitoring rejection status, and in some cases discovering rejection onset well before serious damage occurs to the transplanted organ. While particular embodiments of the invention have been described above, it will be appreciated that other embodiments are also intended to be within the scope and spirit of the invention. For example, one could use a labeled antibody to the marker protein to bind to the sample of interest and provide a more quantitative reading, by comparison of the read-out to a standard curve developed by using the antibody against samples of known condition.

Moreover, the decrease in the phosphorylated selenium binding protein correlates to undesirable proliferation of smooth muscle. This condition can be present in patients who have not experienced transplanting, where there are certain other disease states/adverse conditions associated with smooth muscle abnormalities, such as asthma and atherosclerosis.

Hence, the downregulation or other expression modification of selenium binding protein type proteins should also serve as an indicator for these other states. For example, one could take a biopsy of lung tissue or arterial tissue and examine the levels of phosphorylated SBP-1 protein as compared to normal knowns.

Similarly, SBP-1 is almost identical at the amino acid level to SBP-2 (also known as acetaminophen-binding protein AP56). These proteins differ only in 14 amino acids that are located sporadically along the proteins sequence. SBP-2 mRNA has been shown to be expressed primarily in the liver. The immunohistochemical studies shown here using Ab that recognize both SBP-1 and SBP-2 did not reveal staining of cells other than SMC in liver tissues, suggesting that SPB-2 could well also be SMC-specific. Hence, monitoring that protein should also be productive.

The invention is also not to be limited by the mechanism through which the protein works. In this regard, I believe that selenium binding protein-1 type proteins may function as linker proteins connecting signaling pathways with the cytoskeleton. However, confirming the exact purpose of this protein, and the purpose of SBP-2 type proteins, in vivo will require further study.

Thus, the claims should be looked to in order to judge the full scope of the invention.

### INDUSTRIAL APPLICABILITY

The invention provides methods of monitoring undesirable smooth muscle abnormalities, thereby providing a diagonistic tool for certain disease states/adverse conditions (especially transplant rejection).

I claim:

1. A method of monitoring whether an animal is experiencing a disease and/or adverse condition involving smooth muscle cell abnormalities, the method comprising:
analyzing a sample taken from the animal for the degree of presence of a protein selected from the group consisting of:
(a) phosphorylated proteins having at least 95 percent homology to phosphorylated SEQ. ID NO. 1 in a form in which at least a tyrosine of SEQ. ID NO. 1 has been phosphorylated;
(b) phosphorylated proteins having at least 95 percent homology to phosphorylated SEQ. ID NO. 2 in a form in which at least a tyrosine of SEQ. ID NO. 2 has been phosphorylated;
(c) proteins having at least 95 percent homology to SEQ. ID NO. 1; and
(d) proteins having at least 95 percent homology to Seq. ID NO. 2.

2. The method of claim 1, wherein the animal is a primate and the disease is selected from the group consisting of transplant rejection, arteriosclerosis, asthma, pregnancy related complications involving the uterus, and cancer.

3. The method of claim 1, wherein the sample is a portion of a specimen selected from the group consisting of the animal's transplanted organ, the animal's transplanted tissue, the animal's kidney, the animal's uterus, the animal's breast, the animal's lung, the animal's heart and the animal's liver.

4. The method of claim 3, wherein the method further comprises examining protein fragments solubilized from a homogenate of the sample for the presence of a fragment of the selected protein which is between 20 kDa and 80 kDa in size.

5. The method of claim 1, wherein the protein is SEQ. ID NO. 1 in a form in which at least a tyrosine of SEQ. ID NO. 1 has been phosphorylated.

6. A method of monitoring whether a transplant selected from the group consisting of transplanted organs, transplanted tissues, and transplanted cells is being rejected by an animal recipient of the transplant, comprising:
analyzing a sample taken from the recipient for the degree of presence of a protein selected from the group consisting of:
(a) phosphorylated proteins having at least 95 percent homology to phosphorylated SEQ. ID NO. 1 in a form in which at least a tyrosine of SEQ. ID NO. 1 has been phosphorylated;
(b) phosphorylated proteins having at least 95 percent homology to phosphorylated SEQ. ID NO. 2 in a form in which at least a tyrosine of SEQ. ID NO. 2 has been phosphorylated;
(c) proteins having at least 95 percent homology to SEQ. ID NO. 1; and
(d) proteins having at least 95 percent homology to SEQ. ID NO. 2.

7. The method of claim 6, wherein the method comprises examining protein fragments solubilized from a homogenate of the sample for the presence of a fragment of the selected protein which is between 20 kDa and 80 kDa in size.

8. The method of claim 6, wherein the animal is a primate.

9. The method of claim 8, wherein the animal is a human.

10. The method of claim 6, wherein the transplant is a transplanted organ selected from the group consisting of transplanted hearts, transplanted livers, transplanted lungs and transplanted kidneys.

11. The method of claim 6, wherein the sample is a portion of a transplanted organ.

12. The method of claim 6, wherein the sample is a portion of a transplanted kidney.

13. The method of claim 6, wherein the protein is SEQ. ID NO. 1 in a form in which at least a tyrosine of SEQ. ID NO. 1 has been phosphorylated.

14. A phosphorylated protein fragment in a form isolated from other proteins having a size greater than 100 kDa, wherein the protein is between 20 and 80 kDa in size and is selected from the group consisting of a fragment of phosphorylated SEQ. ID NO. 1 in a form in which at least a tyrosine of SEQ. ID NO. 1 has been phosphorylated and a fragment of phosphorylated SEQ. ID NO. 2 in a form in which at least a tyrosine of SEQ. ID NO. 2 has been phosphorylated.

15. An antibody capable of binding to at least two of the claim 1 proteins, at least one of which is not phosphorylated, and at least one of which is phosphorylated.

16. A kit for monitoring whether an animal is experiencing a disease and/or adverse condition involving smooth muscle cell abnormalities, the kit comprising a claim 15 antibody.


SEQUENCE LISTING (2 sequences)

SEQ ID NO: 1 - protein, 472 aa, Homo sapiens
MATKCGNCGPGYSTPLEAMKGPREEIVYLPCIYRNTGTEAPDYLATVDVDPKSPQYCQVIHRLPMPNLKDELHHSGWNTCSSCFGDSTKSRTKLVLPSLISSRIYVVDVGSEPRAPKLHKVIEPKDIHAKCELAFLHTSHCLASGEVMISSLGDVKGNGKGGFVLLDGETFEVKGTWERPGGAAPLGYDFWYQPRHNVMISTEWAAPNVLRDGFNPADVEAGLYGSHLYVWDWQRHEIVQTLSLKDGLIPLEIRFLHNPDAAQGFVGCALSSTIQRFYKNEGGTWSVEKVIQVPPKKVKGWLLPEMPGLITDILLSLDDRFLYFSNWLHGDLRQYDISDPQRPRLTGQLFLGGSIVKGGPVQVLEDEELKSQPEPLVVKGKRVAGGPQMIQLSLDGKRLYITTSLYSAWDKQFYPDLIREGSVMLQVDVDTVKGGLKLNPNFLVDFGKEPLGPALAHELRYPGGDCSSDIWI

SEQ ID NO: 2 - protein, 472 aa, Rattus norvegicus
MATKCTKCGPGYATPLEAMKGPREEIVYLPCIYRNTGIEAPDYLATVDVDPKSPHYSQVIHRLPMPHLKDELHHSGWNTCSSCFGDSTKSRDKLILPSIISSRIYVVDVGSEPRAPKLHKVIEPNEIHAKCNLGNLHTSHCLASGEVMISSLGDPQGNGKGGFVLLDGETFEVKGTWEKPGGEAPMGYDFWYQPRHNIMVSTEWAAPNVFKDGFNPAHVEAGLYGSHIHVWDWQRHEIIQTLQMKDGLIPLEIRFLHDPDATQGFVGCALSSNIQRFYKNEGGTWSVEKVIQVPSKKVKGWMLPEMPGLITDILLSLDDRFLYFSNWLHGDIRQYDISNPKKPRLTGQIFLGGSIVKGGSVQVLEDQELTCQPEPLVVKGKRVPGGPQMIQLSLDGKRLYVTTSLYSAWDKQFYPNLIREGSVMLQIDVDTANGGLKLNPNFLVDFGKEPLGPALAHELRYPGGDCSSDIWI